SEQUENCE LISTING (2 sequences)

SEQ ID NO: 1 - RNA, 1404 nt, synthetic construct
  source 1..1404: mol_type = other RNA
atggagacagacacactgcttctgtgggtgctgctcttgtgggtgcctggcagcaccggggatgaggtgcagctggtgcagagcggcgccgaggtgaagaagcccggcgccagcgtcaaggtcagctgcaaggccagcgggtacaagttcaccgacagtgagatgcactgggtgaggcaggcccctggccaggggctggagtggataggcggcgtggaccccgaaaccgagggcgccgcctacaaccagaagttcaagggcagggccaccatcaccagggatacctccacaagcaccgcttacctcgagctgtcaagcttgaggtcggaggataccctggccctggtgtactactgcaccagggggtacgactacgactatgccctggactactggggacagggcaccttggtgaccgtgtccagcgcgagcaccaagggcccttccgtgttccctctggcccctagcagcaagagcaccagcggcggcaccgccgccctgggctgcctggtgaaggattactttcctgagccagtgaccgtgagctggaactccggcgctctgaccagcggcgtgcacaccttccccgccgtgctgcagagcagcgggctgtacagcctgagcagcgtggtgaccgtgccaagcagctccctgggcacacagacctacatctgtaatgtgaaccacaagccctctaacaccaaggtggacaagagggtggagagcaagtacggccctccttgtcctccttgcccagcccctgaggcagccggcggccctagcgttttcctgttccctcctaagcccaaggacaccctgatgatcagcaggacacctgaggtgacctgcgtcgtggtggacgtgagccaggaggaccctgaggtgcagttcaactggtacgtcgacggcgtggaggtgcacaacgccaagaccaagcctcgagaggagcagttcaacagcacctacagggtggtgagcgtgctgaccgtgctgcaccaggactggctgaacggcaaggagtacaagtgcaaggtgtccaacaagggactgcctagcagcatagagaagaccatctctaaggccaagggccagcccagggagcctcaggtgtacaccctgccccccagccaggaggagatgaccaagaaccaggtgtccctgacctgtctggtcaagggcttctaccccagcgacatcgctgtggagtgggagtccaatggtcagcctgagaacaactacaagaccactcctcctgtgctggactccgacggcagcttcttcctgtatagcaggctgaccgtcgacaagagcaggtggcaggagggcaacgtgttcagctgcagcgtgatgcacgaggccctccacaaccactacacacagaagtcactgagtctgtctctgggc

SEQ ID NO: 2 - RNA, 702 nt, synthetic construct
  source 1..702: mol_type = other RNA
atggaaaccgacacgctcctgctgtgggtgctgttgctgtgggtgcccggcagcaccggtgatgacatccagatgacccagtcccccagcagcctgtcggctagcgtgggggaccgggtcaccatcacctgccgggcatccagcagcgtggagttcatccattggtaccagcagaagcctggcaaggcccctaagcctctgatctccgccactagtaacctggcctccggggtgccgagcaggttctcaggcagcgggtctggcactgatttcaccctgacaatcagcagcctgcagcctgaggacttcgccacctactactgccagcagtggagctcggcaccctggaccttcggccaggggactaaggtggagatcaagaggaccgtggccgccccttcagtgttcatcttccctccttctgatgagcagctgaagtccggcacagcctccgtggtgtgcctgctgaacaacttctaccctagggaggccaaggtgcagtggaaggtggacaacgccctccagagcgggaatagccaggagtctgttaccgagcaggactccaaggacagcacctacagcctgagcagcactctcaccctgtctaaggctgattacgagaagcacaaggtgtacgcttgtgaggtcacccaccaggggctgtcaagccccgtgaccaagagctttaacaggggagagtgc

(19) United States
(12) Patent Application Publication
(10) Pub. No.: US 2026/0137712 A1
(43) Pub. Date: May 21, 2026

(54) MRNA ENCODING HTRA1 PROTEIN ANTIBODY AND APPLICATION THEREOF

(71) Applicant: THE FIRST AFFILIATED HOSPITAL OF DALIAN MEDICAL UNIVERSITY, Dalian City, Liaoning Province (CN)

(72) Inventors: Xufeng TAO, Dalian City (CN); Deshi DONG, Dalian City (CN); Hong XIANG, Dalian City (CN); Xinya ZHAO, Dalian City (CN); Linlin LYU, Dalian City (CN); Xin KONG, Dalian City (CN)

(21) Appl. No.: 19/205,243

(22) Filed: May 12, 2025

(30) Foreign Application Priority Data
Nov. 15, 2024 (CN) 202411632731.8

(51) Int. Cl. A61K 31/7105; A61K 9/127; A61K 9/51; A61P 35/00; C07K 16/40; C12N 15/88

(52) U.S. Cl. CPC A61K 31/7105; A61K 9/127; A61K 9/5123; A61P 35/00; C07K 16/40; C12N 15/88

(57) ABSTRACT

An mRNA encoding an HTRA1 protein antibody and an application thereof are provided. The mRNA encoding the HTRA1 protein antibody is provided, including an mRNA encoding a heavy chain of the HTRA1 protein antibody with a sequence shown in SEQ ID No. 1 and an mRNA encoding a light chain of the HTRA1 protein antibody with a sequence shown in SEQ ID No. 2.

### CROSS-REFERENCE TO RELATED APPLICATIONS

This application claims priority to Chinese Patent Application No. 202411632731.8, filed on Nov. 15, 2024, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: 207009-1-PTO-20250512-sequence_listing.xml
Creation date: 2024 Nov. 8
Byte size: 4,811

### TECHNICAL FIELD

The present disclosure belongs to the technical field of biopharmaceuticals, and in particular relates to a messenger ribonucleic acid (mRNA) encoding high temperature requirement factor A1 (HTRA1) protein antibody and an application thereof.

### BACKGROUND

Pancreatic cancer (PC) is a malignant tumor with an extremely poor prognosis, with a 5-year survival rate of only 12%, earning it the title of the “king of cancers.” It is projected that by 2030, pancreatic cancer may become the second leading cause of cancer-related deaths. The pancreas is located in a concealed position, and early-stage symptoms are nonspecific, making them easy to overlook. This increases the difficulty of early diagnosis for pancreatic cancer. At the time of initial diagnosis, only 15%-20% of patients are eligible for surgical resection. However, due to the high malignancy of pancreatic cancer, the postoperative recurrence rate is as high as 85%, and these recurrent patients often exhibit high resistance to radiotherapy and chemotherapy medicines. Therefore, standardized and rational medicine treatment for pancreatic cancer patients undergoing surgery is of great significance in improving surgical outcomes, prolonging patient survival, and enhancing quality of life.

HTRA1 is a heat shock-induced, envelope-associated serine protease with dual activities of binding molecular chaperones and proteases, capable of recognizing and degrading misfolded proteins in the cytoplasm. As a secretory protein, HTRA1 is involved in the degradation of the extracellular matrix. In addition to serving as a tumor marker and/or prognostic factor, HTRA1 is also associated with tumorigenesis by regulating tumor cell proliferation, migration, apoptosis, and differentiation.

Currently, antibody-based medicines have made rapid progress in the biopharmaceutical field. However, the fragile nature of monoclonal antibodies and the high costs associated with their production, storage, transportation, and distribution limit their global application. Nucleic acid-encoded monoclonal antibodies, particularly mRNA-based monoclonal antibodies, offer great promise for improving antibody therapy outcomes. mRNA therapeutics leverage the natural process of intracellular protein synthesis for treatment. By encoding immunostimulatory molecules or antigens, mRNA medicines may activate the immune system to target and destroy pathogens or cancer cells, thereby achieving disease prevention and treatment. The mRNA therapy is a novel form of gene therapy. Synthetic mRNA provides a template for the synthesis of any given protein, protein fragment, or peptide, facilitating a wide range of pharmaceutical applications, including various modes of cancer immunotherapy. mRNA therapy not only mediates superior transfection efficiency and longer protein expression duration but also holds significant advantages over DNA. Currently, there are no relevant studies on mRNA medicines targeting HTRA1 for the treatment of pancreatic cancer.

### SUMMARY

An objective of the present disclosure is to provide an mRNA encoding an HTRA1 protein antibody and an application thereof, aiming to address the issues present in the prior art. The mRNA encoding the HTRA1 protein antibody provided by the present disclosure may inhibit the occurrence and progression of pancreatic cancer by targeting the expression of HTRA1 in cells.

To achieve the above objective, the present disclosure provides the following solutions.

The present disclosure provides an mRNA encoding an HTRA1 protein antibody, where the mRNA includes an mRNA encoding a heavy chain of the HTRA1 protein antibody and an mRNA encoding a light chain of the HTRA1 protein antibody.

The mRNA encoding the heavy chain of the HTRA1 protein antibody has a sequence as shown in SEQ ID No. 1, and the mRNA encoding the light chain of the HTRA1 protein antibody has a sequence as shown in SEQ ID No. 2.

The present disclosure provides a pharmaceutical composition, where the pharmaceutical composition includes the mRNA and a delivery vector.

Optionally, the delivery vector includes lipid nanoparticles.

Optionally, the pharmaceutical composition further includes pharmaceutically acceptable excipients.

The pharmaceutically acceptable excipients include one or more of fillers, diluents, binders, disintegrants, emulsifiers, and non-toxic medicine vectors.

Optionally, a dosage form of the pharmaceutical composition includes an injectable preparation.

Further optionally, the present disclosure provides an application of the mRNA or the pharmaceutical composition in preparing an mRNA medicine.

Further optionally, the present disclosure provides an mRNA medicine, where the medicine includes the mRNA.

Further optionally, the medicine further includes pharmaceutically acceptable excipients.

The pharmaceutically acceptable excipients include one or more of fillers, diluents, binders, disintegrants, emulsifiers, and non-toxic medicine vectors.

Further optionally, a dosage form of the medicine includes an injectable preparation.

The present disclosure provides an application of the mRNA or the pharmaceutical composition in preparing a medicine for treating pancreatic cancer.

Optionally, the medicine achieves a therapeutic effect on pancreatic cancer by inhibiting an expression of HTRA1 protein.

The present disclosure provides a medicine for treating pancreatic cancer, where the medicine includes the mRNA or the pharmaceutical composition.

Optionally, the medicine further includes pharmaceutically acceptable excipients.

The pharmaceutically acceptable excipients include one or more of fillers, diluents, binders, disintegrants, emulsifiers, and non-toxic medicine vectors.

Optionally, a dosage form of the medicine includes an injectable preparation.

The present disclosure discloses the following technical effects.

Specific embodiments of the present disclosure employ the mRNA encoding the HTRA1 protein antibody (Anti-HTRA1 mRNA) for in vitro experiments, demonstrating that Anti-HTRA1 mRNA may inhibit the expression of the HTRA1 protein in pancreatic cells, while limiting the proliferation, invasion, adhesion, and migration of PANC-1 and SW1990 cells, promoting apoptosis of PANC-1 and SW1990 cells. Thus, the Anti-HTRA1 mRNA provided by the present disclosure may be used for cancer treatment, particularly pancreatic cancer. The present disclosure provides a basis for utilizing Anti-HTRA1 mRNA as a novel medicine for treating pancreatic cancer.

### BRIEF DESCRIPTION OF THE DRAWINGS

In order to explain the embodiments of the present disclosure or the technical solution in the prior art more clearly, the drawings needed in the embodiments will be briefly introduced below. Apparently, the drawings in the following description are only some embodiments of the present disclosure. For one of ordinary skill in the art, other drawings may be obtained according to these drawings without creative effort.

FIG. 1A is a Western blot detection image of HTRA1 protein expression in PANC-1 and SW1990 cells.

FIG. 1B is a statistical graph of HTRA1 protein expression levels in PANC-1 and SW1990 cells. Data are presented as mean±standard error of mean (SEM). Compared to Control (Ctrl), ** represents P<0.01.

FIG. 2 shows results of cell counting kit-8 (CCK-8) detection of cell viability in PANC-1 and SW1990 cells. Data are presented as mean±SEM. Compared to Ctrl, * represents P<0.05, and ** represents P<0.01.

FIG. 3A shows results of Transwell experiment to detect invasion ability of PANC-1 and SW1990 cells.

FIG. 3B is a statistical graph of invasion rates of PANC-1 and SW1990 cells in the Transwell experiment. Data are presented as mean±SEM. Compared to Ctrl, ** represents P<0.01.

FIG. 4A shows results of adhesion experiment to detect adhesion ability of PANC-1 cells.

FIG. 4B shows results of adhesion experiment to detect adhesion ability of SW1990 cells. Data are presented as mean±SEM. Compared to Ctrl, ** represents P<0.01.

FIG. 5A shows results of scratch experiments to detect migration ability of PANC-1 and SW1990 cells.

FIG. 5B is a statistical graph of cell migration rates of scratch experiments of PANC-1 and SW1990 cells. Data are presented as mean±SEM. Compared to Ctrl, * represents P<0.05, and ** represents P<0.01.

FIG. 6A is a flow cytometry image.

FIG. 6B is a statistical graph of cell apoptosis percentages. Data are presented as mean±SEM. Compared to Ctrl, * represents P<0.05, and ** represents P<0.01.

FIG. 7 shows a secondary structure of in vitro transcription-messenger ribonucleic acid (IVT-mRNA) of Anti-HTRA1 heavy chain.

FIG. 8 shows a secondary structure of IVT-mRNA of Anti-HTRA1 light chain.

### DETAILED DESCRIPTION OF THE EMBODIMENTS

Various exemplary embodiments of the present disclosure are now described in detail. This detailed description should not be construed as limiting the present disclosure but rather as providing a more detailed explanation of certain aspects, features, and implementations of the present disclosure.

It should be understood that the terms used herein are intended only to describe specific embodiments and are not intended to limit the present disclosure. Additionally, for numerical ranges in the present disclosure, it should be understood that each intermediate value between the upper and lower limits of the range is also specifically disclosed. Any stated value or intermediate value within a stated range, as well as any smaller range derived from such intermediate values, is also included in the present disclosure. The upper and lower limits of these smaller ranges may be independently included or excluded from the range.

Unless otherwise specified, all technical and scientific terms used herein have the same meanings as commonly understood by one of ordinary skill in the art to which this present disclosure belongs. Although only preferred methods and materials are described herein, any methods and materials similar or equivalent to those described may also be used in the practice or testing of the present disclosure. All publications cited herein are incorporated by reference to disclose and describe the methods and/or materials related to such publications. In case of conflict with any incorporated publication, the content of this specification shall prevail.

Various modifications and changes may be made to the specific embodiments described herein without departing from the scope or spirit of the present disclosure, as will be apparent to those skilled in the art. Other embodiments derived from the description herein will also be apparent to those skilled in the art. The description and embodiments herein are illustrative only.

As used herein, terms such as “comprising,” “including,” “having,” and “containing” are open-ended and mean “including but not limited to.”

Unless otherwise required, the reagents used in the present disclosure are those conventionally purchased by those skilled in the art, and the methods used are well-known to those skilled in the art.

Previous research by our team found that HTRA1 promotes the release of inflammatory factors by inhibiting transforming growth factor-beta-mediated (TGF-β-mediated) anti-inflammatory responses, exacerbating acute pancreatitis. Further studies revealed that HTRA1 may promote the progression of pancreatic cancer and is a key factor in its development. Therefore, targeted HTRA1 therapy may yield favorable therapeutic effects for pancreatic cancer.

### Embodiment 1: Design and Synthesis of Anti-HTRA1 mRNA

(1) A coding sequence (CDS) region of Anti-HTRA1 mRNA sequence is obtained.
(2) The CDS region of the Anti-HTRA1 mRNA sequence is optimized using artificial intelligence (AI) algorithms.
(3) The IVT-mRNA of the Anti-HTRA1 heavy chain

(SEQ ID No. 1, specifically:
ATGGAGACAGACACACTGCTTCTGTGGGTGCTGCTCTTGTGGGTG
CCTGGCAGCACCGGGGATGAGGTGCAGCTGGTGCAGAGCGGCGCC
GAGGTGAAGAAGCCCGGCGCCAGCGTCAAGGTCAGCTGCAAGGCC
AGCGGGTACAAGTTCACCGACAGTGAGATGCACTGGGTGAGGCAG
GCCCCTGGCCAGGGGCTGGAGTGGATAGGCGGCGTGGACCCCGAA
ACCGAGGGCGCCGCCTACAACCAGAAGTTCAAGGGCAGGGCCACC
ATCACCAGGGATACCTCCACAAGCACCGCTTACCTCGAGCTGTCA
AGCTTGAGGTCGGAGGATACCCTGGCCCTGGTGTACTACTGCACC
AGGGGGTACGACTACGACTATGCCCTGGACTACTGGGGACAGGGC
ACCTTGGTGACCGTGTCCAGCGCGAGCACCAAGGGCCCTTCCGTG
TTCCCTCTGGCCCCTAGCAGCAAGAGCACCAGCGGCGGCACCGCC
GCCCTGGGCTGCCTGGTGAAGGATTACTTTCCTGAGCCAGTGACC
GTGAGCTGGAACTCCGGCGCTCTGACCAGCGGCGTGCACACCTTC
CCCGCCGTGCTGCAGAGCAGCGGGCTGTACAGCCTGAGCAGCGTG
GTGACCGTGCCAAGCAGCTCCCTGGGCACACAGACCTACATCTGT
AATGTGAACCACAAGCCCTCTAACACCAAGGTGGACAAGAGGGTG
GAGAGCAAGTACGGCCCTCCTTGTCCTCCTTGCCCAGCCCCTGAG
GCAGCCGGCGGCCCTAGCGTTTTCCTGTTCCCTCCTAAGCCCAAG
GACACCCTGATGATCAGCAGGACACCTGAGGTGACCTGCGTCGTG
GTGGACGTGAGCCAGGAGGACCCTGAGGTGCAGTTCAACTGGTAC
GTCGACGGCGTGGAGGTGCACAACGCCAAGACCAAGCCTCGAGAG
GAGCAGTTCAACAGCACCTACAGGGTGGTGAGCGTGCTGACCGTG
CTGCACCAGGACTGGCTGAACGGCAAGGAGTACAAGTGCAAGGTG
TCCAACAAGGGACTGCCTAGCAGCATAGAGAAGACCATCTCTAAG
GCCAAGGGCCAGCCCAGGGAGCCTCAGGTGTACACCCTGCCCCCC
AGCCAGGAGGAGATGACCAAGAACCAGGTGTCCCTGACCTGTCTG
GTCAAGGGCTTCTACCCCAGCGACATCGCTGTGGAGTGGGAGTCC
AATGGTCAGCCTGAGAACAACTACAAGACCACTCCTCCTGTGCTG
GACTCCGACGGCAGCTTCTTCCTGTATAGCAGGCTGACCGTCGAC
AAGAGCAGGTGGCAGGAGGGCAACGTGTTCAGCTGCAGCGTGATG
CACGAGGCCCTCCACAACCACTACACACAGAAGTCACTGAGTCTG
TCTCTGGGC)

and the IVT-mRNA of the Anti-HTRA1 light chain

(SEQ ID No. 2, specifically:
ATGGAAACCGACACGCTCCTGCTGTGGGTGCTGTTGCTGTGGGTG
CCCGGCAGCACCGGTGATGACATCCAGATGACCCAGTCCCCCAGC
AGCCTGTCGGCTAGCGTGGGGGACCGGGTCACCATCACCTGCCGG
GCATCCAGCAGCGTGGAGTTCATCCATTGGTACCAGCAGAAGCCT
GGCAAGGCCCCTAAGCCTCTGATCTCCGCCACTAGTAACCTGGCC
TCCGGGGTGCCGAGCAGGTTCTCAGGCAGCGGGTCTGGCACTGAT
TTCACCCTGACAATCAGCAGCCTGCAGCCTGAGGACTTCGCCACC
TACTACTGCCAGCAGTGGAGCTCGGCACCCTGGACCTTCGGCCAG
GGGACTAAGGTGGAGATCAAGAGGACCGTGGCCGCCCCTTCAGTG
TTCATCTTCCCTCCTTCTGATGAGCAGCTGAAGTCCGGCACAGCC
TCCGTGGTGTGCCTGCTGAACAACTTCTACCCTAGGGAGGCCAAG
GTGCAGTGGAAGGTGGACAACGCCCTCCAGAGCGGGAATAGCCAG
GAGTCTGTTACCGAGCAGGACTCCAAGGACAGCACCTACAGCCTG
AGCAGCACTCTCACCCTGTCTAAGGCTGATTACGAGAAGCACAAG
GTGTACGCTTGTGAGGTCACCCACCAGGGGCTGTCAAGCCCCGTG
ACCAAGAGCTTTAACAGGGGAGAGTGC)

are synthesized, obtaining the Anti-HTRA1 mRNA.

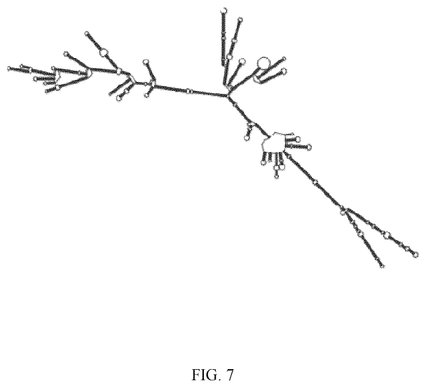

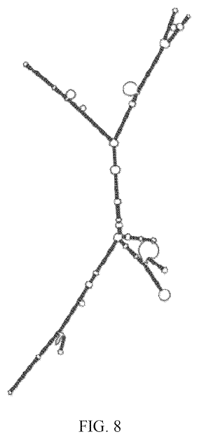

The secondary structure of the IVT-mRNA of the Anti-HTRA1 heavy chain is shown in FIG. 7, and the secondary structure of the IVT-mRNA of the Anti-HTRA1 light chain is shown in FIG. 8.

### Embodiment 2: In Vitro Experiments

### 1. Experimental Methods

JetMESSENGER is used for in vitro transfection. The transfection steps are as follows: PANC-1 or SW1990 cells are inoculated at a density of 1×10⁶ cells per milliliter (cells/mL) in a 100 millimeters (mm) cell culture dish and cultured overnight until 70%-80% fusion is reached.

The cell experiment is divided into two groups: the control group (Ctrl) and the experimental group (Anti-HTRA1 mRNA). After the cells are fused to 70%-80%, 5 microgram (μg) of the IVT-mRNA of the Anti-HTRA1 heavy chain (SEQ ID No. 1) and 5 μg of the IVT-mRNA of the Anti-HTRA1 light chain (SEQ ID No. 2) are added to a 1000 microliter (μL) mRNA buffer, blown and mixed well. Then, 20 μL of jetMESSENGER reagent is added, blown and mixed well, and the mixture is allowed to stand at room temperature for 10 minutes (min) to obtain Anti-HTRA1 mRNA complex. Among them, jetMESSENGER transfection reagent is added to Ctrl group, and Anti-HTRA1 mRNA complex is added to Anti-HTRA1 mRNA group. 5 milliliter (mL) of Dulbecco's modified eagle medium (DMEM) high-glucose medium is added to the cell culture dishes of the Ctrl group and the Anti-HTRA1 mRNA group. After mixing well, the mixture is incubated in an incubator for 24 hours (h). Subsequently, the efficacy of Anti-HTRA1 mRNA medicine is validated through Western blot, CCK-8 cell proliferation assay, cell invasion assay, cell adhesion assay, and flow cytometry detection.

### 2. Experimental Results

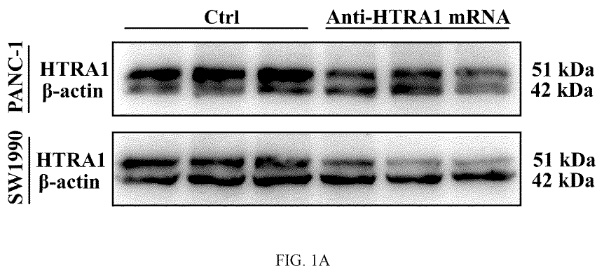

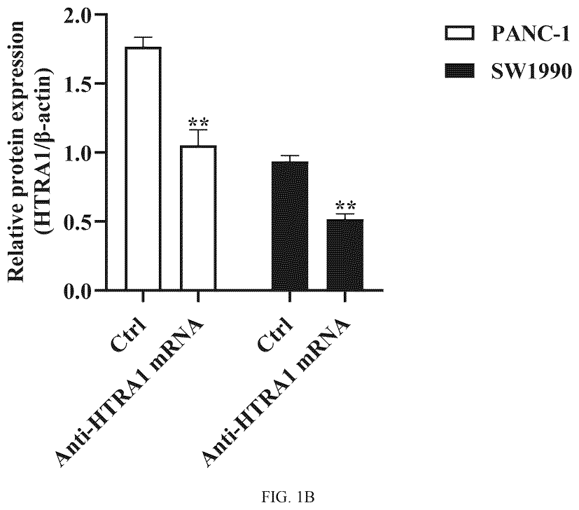

Western blot results are shown in FIG. 1A-FIG. 1B. After transfection with Anti-HTRA1 mRNA (IVT-mRNA of the Anti-HTRA1 heavy chain and IVT-mRNA of the Anti-HTRA1 light chain), HTRA1 protein levels in PANC-1 and SW1990 cells are significantly downregulated.

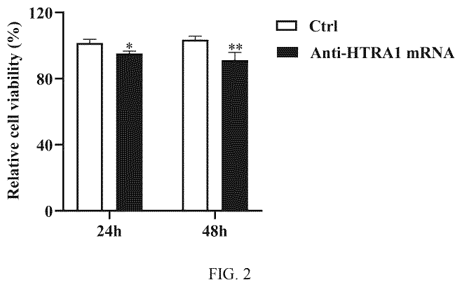

The CCK-8 assay is used to detect the effect of medicines on cell proliferation. The results show that after transfection with Anti-HTRA1 mRNA for 24 h and 48 h, the cell viability of PANC-1 and SW1990 cells decreases, indicating that Anti-HTRA1 mRNA medicines inhibit the cell viability of PANC-1 and SW1990 cells (FIG. 2).

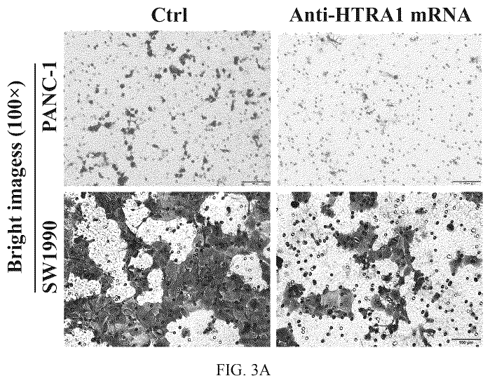

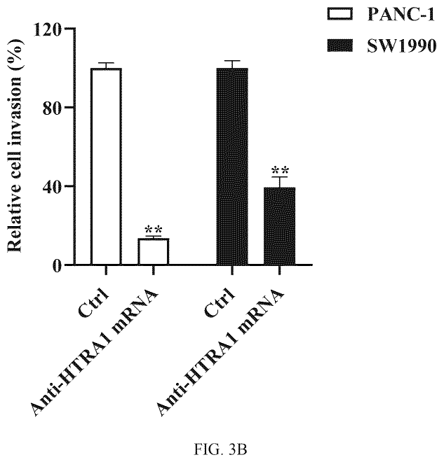

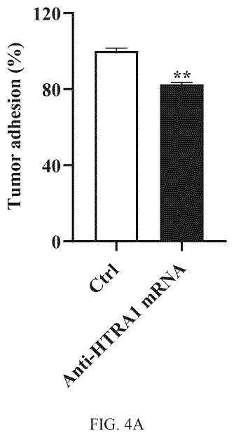

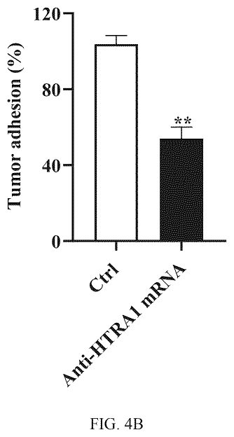

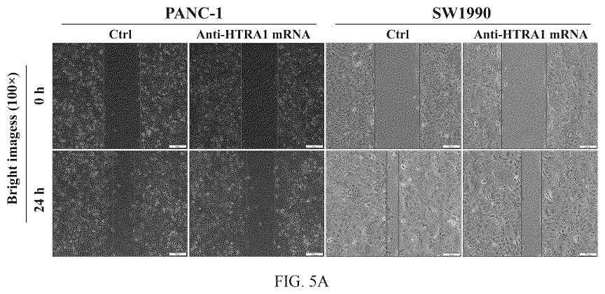

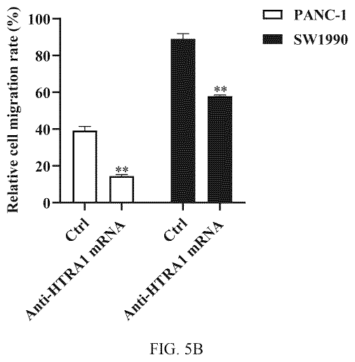

The results of Transwell, adhesion and scratch experiments also reveal that transfection of Anti-HTRA1 mRNA in PANC-1 and SW1990 cells significantly reduces the number of invasive cells, cell adhesion activity, and cell migration ability (FIG. 3A, FIG. 3B, FIG. 4A, FIG. 4B, FIG. 5A, FIG. 5B).

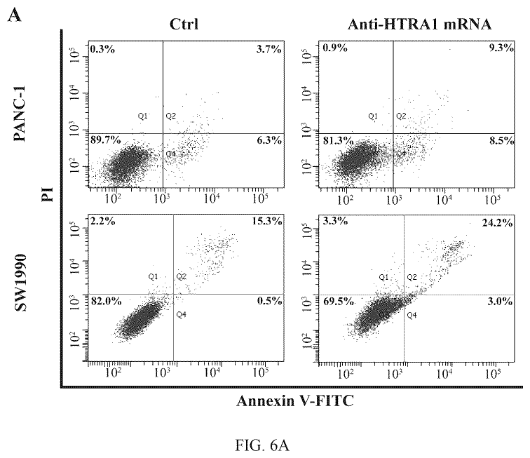

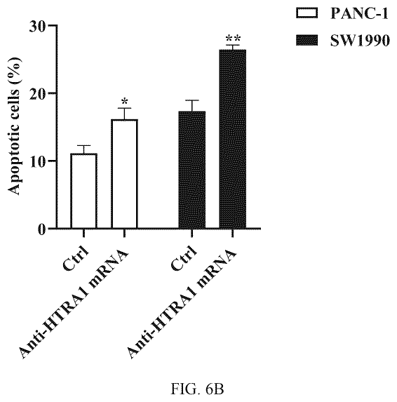

In addition, flow cytometry shows that the total proportion of cells in Q2 and Q4 is significantly higher in the Anti-HTRA1 mRNA treatment compared to the Ctrl group (FIG. 6A-FIG. 6B). These results confirm that Anti-HTRA1 mRNA inhibits proliferation, migration, invasion, and adhesion of PANC-1 and SW1990 cells while promoting apoptosis.

### Embodiment 3

Preparation of medicines containing Anti-HTRA1 mRNA: the IVT-mRNA of Anti-HTRA1 heavy chain and the IVT-mRNA of Anti-HTRA1 light chain are mixed with lipid single nucleotide polymorphism (SNP) to obtain lipid nanoparticles (SNP-encapsulated lipid nanoparticles), thus obtaining medicines containing Anti-HTRA1 mRNA.

The embodiments described above are merely illustrative of the preferred modes of the present disclosure and do not limit the scope of the present disclosure. Without departing from the spirit of the present disclosure, any modifications or improvements made by those skilled in the art to the technical solutions described herein shall fall within the scope defined by the claims of the present disclosure.

What is claimed is:

1. An mRNA encoding an HTRA1 protein antibody, wherein the mRNA comprises an mRNA encoding a heavy chain of the HTRA1 protein antibody and an mRNA encoding a light chain of the HTRA1 protein antibody;
wherein the mRNA encoding the heavy chain of the HTRA1 protein antibody has a sequence as shown in SEQ ID No. 1, and the mRNA encoding the light chain of the HTRA1 protein antibody has a sequence as shown in SEQ ID No. 2.

2. A pharmaceutical composition, wherein the pharmaceutical composition comprises the mRNA according to claim 1 and a delivery vector.

3. The pharmaceutical composition according to claim 2, wherein the delivery vector comprises lipid nanoparticles.

4. The pharmaceutical composition according to claim 2, wherein the pharmaceutical composition further comprises pharmaceutically acceptable excipients;
wherein the pharmaceutically acceptable excipients comprise one or more of fillers, diluents, binders, disintegrants, emulsifiers, and non-toxic medicine vectors.

5. The pharmaceutical composition according to claim 4, wherein a dosage form of the pharmaceutical composition comprises an injectable preparation.

6. A medicine for treating pancreatic cancer, wherein the medicine comprises the mRNA or the pharmaceutical composition according to claim 2.

7. The medicine according to claim 6, wherein the medicine further comprises pharmaceutically acceptable excipients;
wherein the pharmaceutically acceptable excipients comprise one or more of fillers, diluents, binders, disintegrants, emulsifiers, and non-toxic medicine vectors.

8. The medicine according to claim 7, wherein a dosage form of the medicine comprises an injectable preparation.
